(19) United States
(12) Patent Application Publication
(10) Pub. No.: US 2019/0175045 A1
(43) Pub. Date: Jun. 13, 2019

(54) Neuromuscular Training Apparatus and Technique

(71) Applicant: Frank Edward Joutras, Lincoln, NE (US)

(72) Inventor: Frank Edward Joutras, Lincoln, NE (US)

(21) Appl. No.: 15/836,265

(22) Filed: Dec. 8, 2017

(51) Int. Cl. A61B 5/0488; A61B 5/00; A61B 5/11

(52) U.S. Cl. CPC A61B 5/0488; A61B 5/6828; G06F 3/015; A61B 5/681; A61B 5/1107

(57) ABSTRACT

To provide a wearable neuromuscular training apparatus comprised of a rigid sections that is worn on a user's left leg and provides asymmetric resistance to the movement of the leg. Said apparatus is designed to address neuromuscular activation patterns that can result in right side dominance, through activating and inhibiting key muscle groups on the body, by providing a programmed neuromuscular resistance pattern to movement while the user performs certain movements or activities. The wearable training device includes an upper rigid section and a lower rigid section that easily and quickly attach to various resistance core sections. Once connected to a resistance core section, the upper and lower sections of the exoskeleton are placed, respectively, above and below a user's knee and attach to the user's leg using strapping or other wearable material. The apparatus provides a controllable range of resistance to the motion of the user's knee. The apparatus can, optionally, include an electronic use management system that can provide guidance and feedback to the user on the correct and efficient utilization of the apparatus. Such guidance and feedback may be provided directly to the user by the apparatus as well as utilizing and interfacing with external computational devices such as smartwatches, smartphones, tablets and computers.

### BACKGROUND OF THE INVENTION

This invention relates to apparatuses and methods for providing neuromuscular training to influence proper biomechanical balance and gait.

In the preferred embodiment, a controlled resistance is provided as part of an attachment system that is worn on the left leg only. A unique type of concentric resistance program provided along with the method of application, result in isolation of key muscles during function that control; the position of the left pelvis, the length-tension ratio of key muscles, changes in gait, and changes in the musculoskeletal system throughout the body.

Conventional equipment like weights, springs, or elastic bands in the related field are not attached to the left leg and use a combination of concentric and eccentric resistance, meaning as resistance is applied when the user's muscle is contracting (concentric) and extending (eccentric). This type of equipment is non-functional, and cannot isolate and inhibit key muscle groups through gait activities.

The resistance provided with conventional training equipment cause both a concentric and eccentric contraction that cannot be isolated during gait, for instance, after moving the leg through flexion and the leg reverses direction into extension, than due to weight or spring/elastic band action, an eccentric resistance is applied during extension to unwanted muscle groups through the range of motion.

Gravity based or spring/rubber band device may cause these unwanted contractions in the muscles. These devices are examples of typical exercise equipment used by health and fitness centers, gyms, or for sports specific training. This is obviously a serious problem to the general users, when users believe they are getting in better condition, but could be causing major muscle Imbalance issues. These imbalances result in reduced functional strength, speed, and agility, along with possible discomfort and risk of injury depending on the severity.

Another method of controlling pelvic position is through manipulation or adjustment of the skeletal system of the body through chiropractic techniques. These techniques, while they may give temporary relief of pain by correcting the skeletal position, do nothing to isolate the muscles that actually hold the skeletal system in proper position. This is why so many patients need to keep returning to the chiropractor for continued manipulation or adjustment.

Prior Art devices that do attach to limbs of the body have several disadvantages, including; resistance that causes an eccentric contraction, resistance that does not provide the correct intensity at specific positions through the range of motion, an attachment system that is bulky and not easy to attach quickly, a frame that does not allow for different attachment shells to be easily interchanged with the central hinge system, a system that does not provide real-time monitoring and recording of position, a system that does not alert the user based on being in the correct or incorrect position, a system that cannot sent wireless signals to a remote device such as a smartwatch, smartphone, tablet, or laptop.

### SUMMARY OF THE INVENTION

It is an object of the invention to provide a novel apparatus and technique for neuromuscular reeducation and gait training by activating key muscle groups while inhibiting over activation of opposing muscle groups.

It is a further object of the invention to provide a novel neuromuscular training technique by which a neuromuscular device is attached only to the left leg to isolate key muscles that control the position of the left pelvis, therefore influencing the musculoskeletal system throughout the human body.

It is a further object of the invention to provide a novel neuromuscular training device that through isolated muscle activation, influences proper gait.

It is a further object of the invention to provide a novel neuromuscular training device that through isolated muscle activation, influences proper muscle length-tension ratios.

It is a further object of the invention to provide a novel neuromuscular training device that includes the ability to easily interchange various sized attachment shells with a central hinged resistance core to allow for multiple different sized users that can share one resistance core. This prevents attachment shells that touch the skin of one user to touch the skin of another user. This prevents transmission of bacteria or transferable diseases from one user to another.

It is a further object of the invention to provide a novel neuromuscular training device that includes the ability to transmit body position signals real-time to the user through a remote device that indicates if the user is in the correct position or not to perform muscle isolation exercises.

It is a further object of the invention to provide a novel neuromuscular training device that includes the ability to transmit body position signals real-time to the user through a remote device that indicates if the user is in the correct position or not while performing gait activities.

It is still a further object of the invention to provide a novel neuromuscular training device that provides haptic and visual alerts to the user based on the position they are in, the number of reps or steps completed, and the speed of movement.

It is still a further object of the invention to provide a novel neuromuscular training device that has a magnetic clip system for quick attachment or removal of the attachment shells to the user.

It is still a further object of the invention to provide a novel neuromuscular training device that only attaches to the left leg to influence proper biomechanical position throughout the whole body.

It is still a further object of the invention to provide a neuromuscular training device that uses sensed position data and a computer processor on the device to vary or cut off resistance to movement based on preset leg position parameters. This insures the user avoids over activation of unwanted muscles during motion which can cause a biomechanical imbalance.

It is still a further object of the invention to provide a neuromuscular training device that provides concentric resistance between 0.5 to 20 ft/lbs in the flexion direction of bending the leg at the knee joint, while at the same time providing concentric resistance between 0 to 5 ft/lbs in the extension direction while bending the leg at the knee joint.

It is a further object of the invention to provide a neuromuscular training device where the resistance is applied through an exoskeleton means that allows complete freedom of functional movements, applies only resistance to concentric muscle contraction that is varied based on a neuromuscular program curve, and the resistance to movement is not affected by speed of movement or gravity, which allows for the absolute isolation of key muscles while inhibiting others.

It is still a further object of the invention to provide a neuromuscular training device that allows for the user's progress to be monitored and tracked through a software application displayed on a remote device such as a smartwatch, smartphone, or other computing devices.

It is still a further object of the invention to provide a neuromuscular training device ice has the ability to receive and transmit data wirelessly to/from the exoskeleton. This allows the user to not be restricted by wires exiting the exoskeleton to other devices while in use.

It is still a further object of this invention to provide a neuromuscular training device within health and fitness centers, gyms, etc. With the new embodied multiple use device, multiple customers can now use the device during warm-up or cool-down to put their body in a balanced position prior to, or after, the use of the gym equipment, which can cause the muscle imbalance. The Sectioned Nature (along with quick swap in/out) with the Upper and Lower Sections being only sections that touch user and that they belong or are assigned to individuals.

It is still a further object of this invention to provide a neuromuscular training device that the resistance core section uses optical or electrical connectors to optically or electronically relay visual responses on exoskeleton sections when connected to the core.

### SUMMARY OF THE DRAWINGS

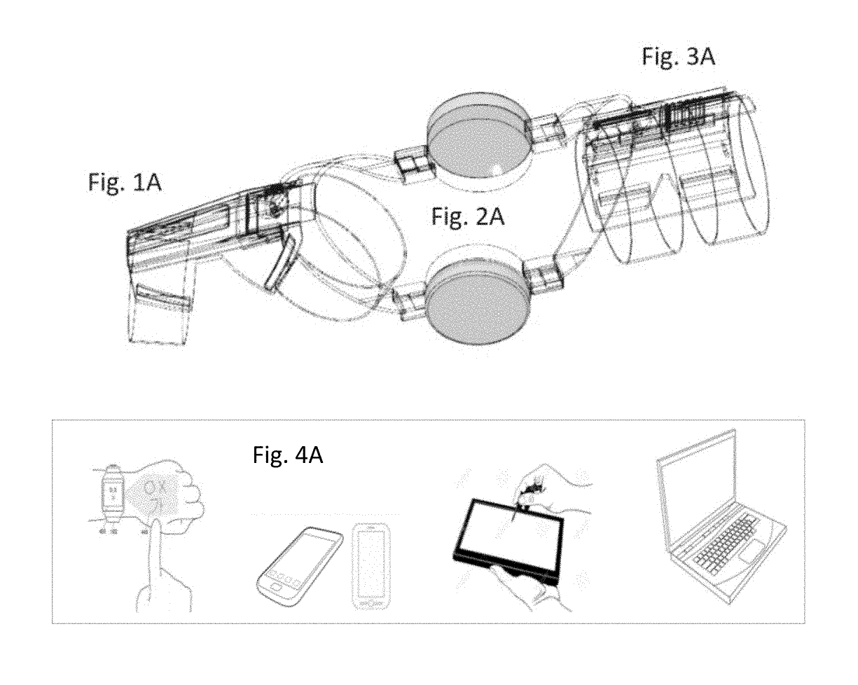

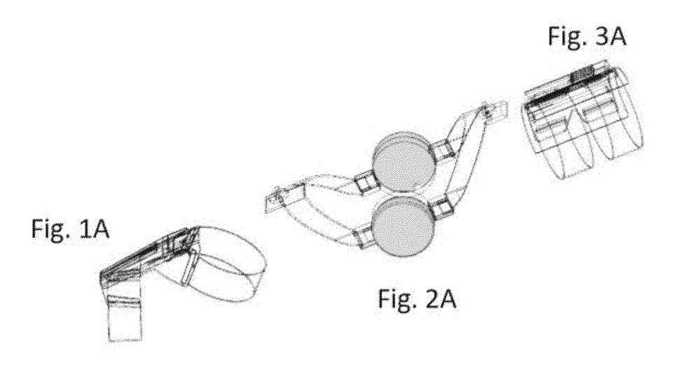

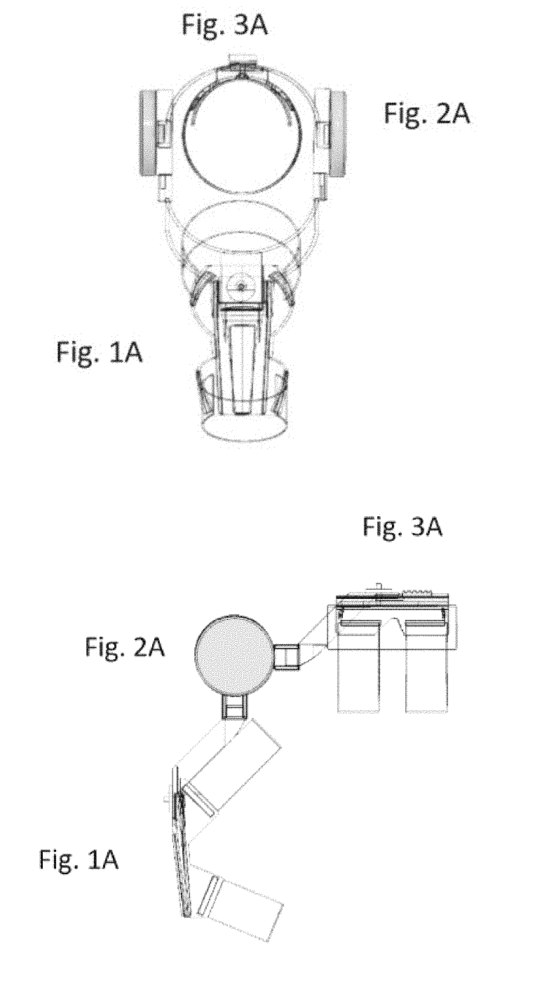

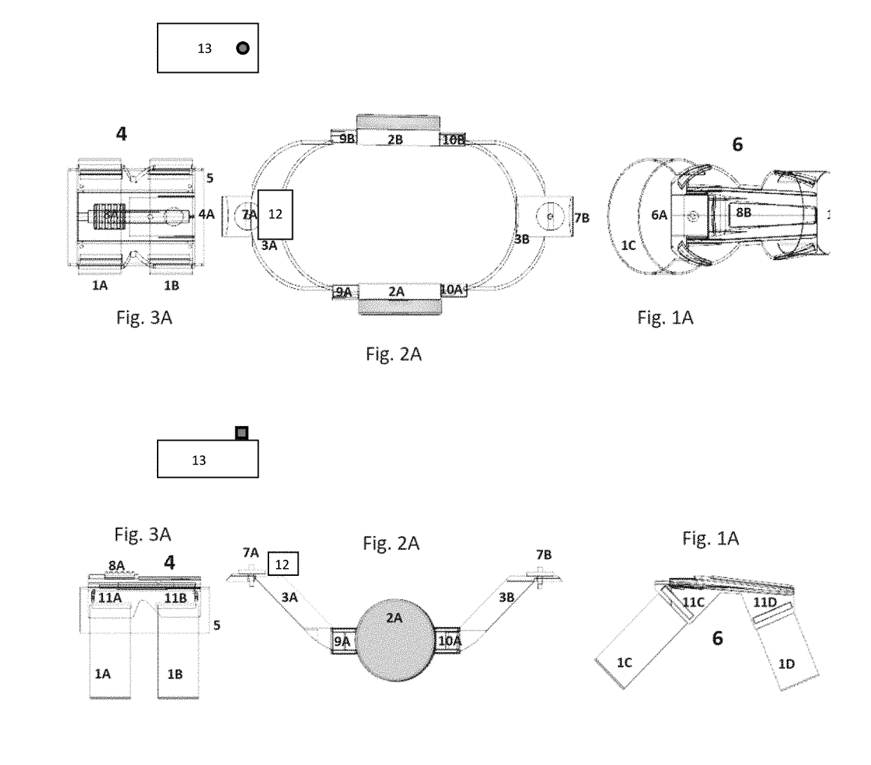

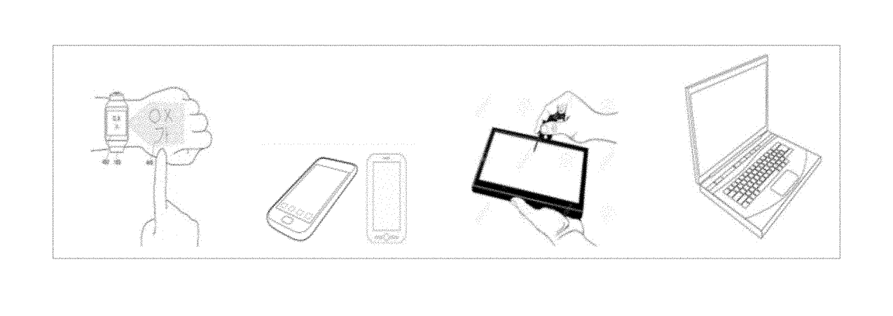

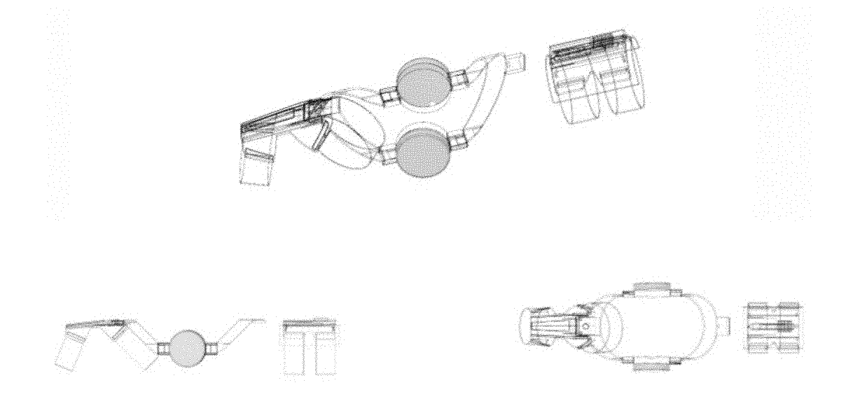

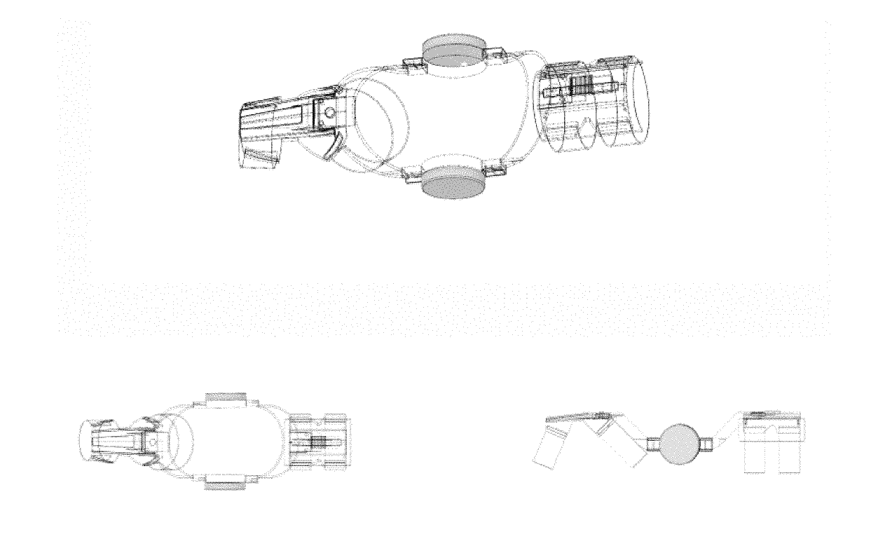

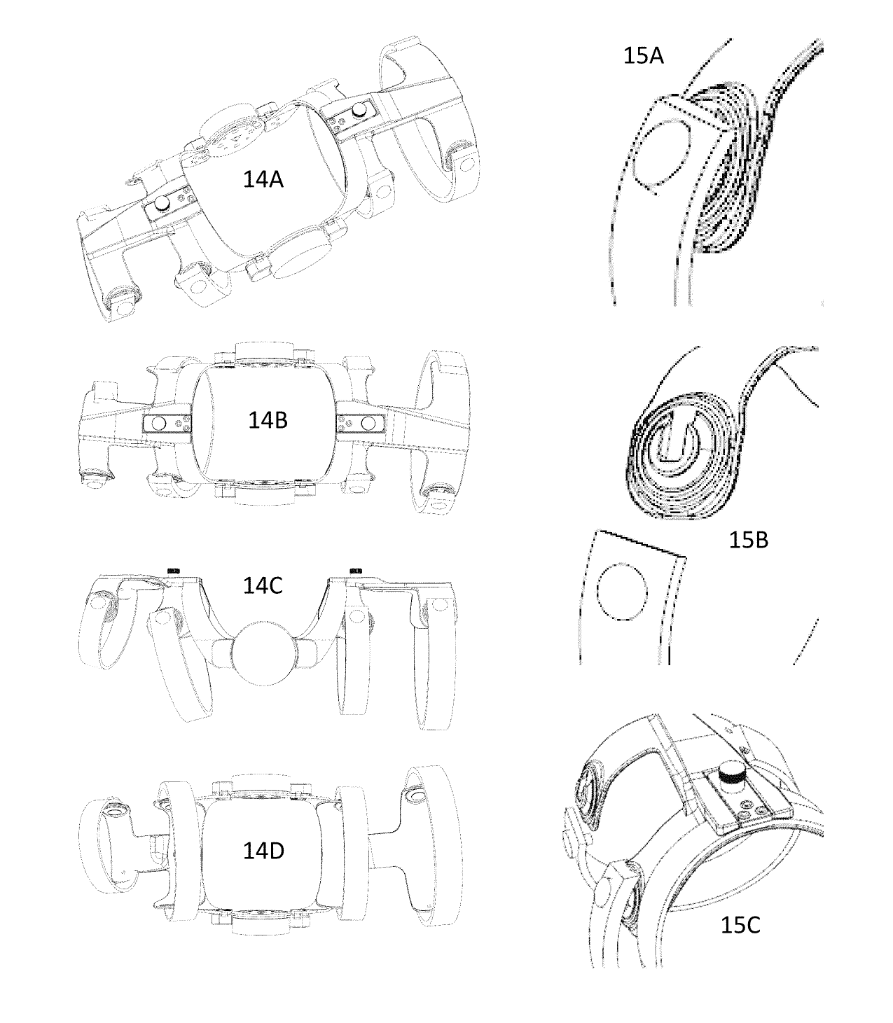

Sheet 1—Shows a 3D view of the Assembled Apparatus including subsections FIG. 1A (Lower section), FIG. 2A (Core), FIG. 3A (Upper Section), FIG. 4A (Remote Devices)

Sheet 2—Shows a 3D view of the Unassembled Apparatus including subsections FIG. 1A (Lower section), FIG. 2A (Core), and FIG. 3A (Upper Section)

Sheet 3—Shows a 2D view of the Assembled Apparatus bent at 90 degrees including subsections FIG. 1A (Lower section), FIG. 2A (Core), and FIG. 3A (Upper Section). There is also a 2D side view of the Assembled Apparatus bent at 90 degrees including subsections FIG. 1A (Lower section), FIG. 2A (Core), and FIG. 3A (Upper Section)

Sheet 4—Shows a 2D top view of the Unassembled Apparatus including subsections FIG. 1A (Lower section), FIG. 2A (Core), FIG. 3A (Upper Section) & individual components 1a, 1b, 1c, 1d—Straps 2a, 2b—Resistance modules 3a, 3b—Crossbars 4, 4a—Upper section, upper section attachment mechanism 5—Sleeve 6, 6a—Lower section, lower section attachment mechanism 7a, 7b—Crossbar attachment mechanisms 8a, 8b—Lights 9a, 9b—Resistance adjustment control 10a, 10b—Position tracking sensors 11a, 11b, 11c, 11d—Strap attachment systems 12—Electronic box with power source, computer processor, and wireless transmitter/receiver 13—Recharging system for power source. In another view, it also shows a 2D side view of the Unassembled Apparatus including subsections FIG. 1A (Lower section), FIG. 2A (Core), FIG. 3A (Upper Section) & individual components 1a, 1b, 1c, 1d—Straps 2a, 2b—Resistance modules 3a, 3b—Crossbars 4, 4a—Upper section, upper section attachment mechanism 5—Sleeve 6, 6a—Lower section, lower section attachment mechanism 7a, 7b—Crossbar attachment mechanisms 8a, 8b—Lights 9a, 9b—Resistance adjustment control 10a, 10b—Position tracking sensors 11a, 11b, 11c, 11d—Strap attachment systems 12—Electronic box with power source, computer processor, and wireless transmitter/receiver 13—Recharging system for power source

Sheet 5—Shows various remote devices that FIG. 2A (Resistance Core) can connect wirelessly or directly. In this description, we show a smart watch smartphone, tablet, and laptop.

Sheet 6—is an alternate embodiment that shows a two-piece connecting system, whereas the resistance source is already part of one section and it connects to the other section to form a moveable exoskeleton for the limb.

Sheet 7—is an alternate embodiment that shows a single piece system that has an upper and lower section and the resistance means to prevent movement between the two sections.

Sheet 8—shows an updated version of the apparatus in views 14A, 14B, 14C, and 14D that includes rigid structures only designed to fit a left leg. In addition, views 15A, 15B, and 15C show an up close view of an alternate attachment which includes magnetic clip attachments for the rigid shells with straps, and an up close view of the male magnetic clip attached to the strap, and the receiving female magnetic clip attached to the shell.

### DETAILED DESCRIPTION OF THE DRAWINGS

The described embodiment includes three sections (FIG. 1A Lower Section, 2A Resistance Core, 3A Upper Section), in addition to individual components of each section. To use the apparatus, FIG. 1A (Lower section) is attached to 2A (Resistance core), then FIG. 3A (Upper Section) is attached to FIG. 2A (Resistance Core). Then components 1C and 1D are wrapped around the user's calf and attached through attachment mechanisms 11C and 11D. Then components 1A and 1B (straps) are wrapped around the user's thigh and attached using attachment mechanisms 11A and 11B. In the preferred embodiment, user selects a software application from FIG. 4A (Smart Watch), which then transmits wireless signals to the resistance core (FIG. 2A), which in turn utilizes the position sensors through Component 12 (computer processor). Based on movement of FIG. 2A (Resistance Core), signals are transmitted back to FIG. 4A (Smart Watch) to create a vibratory response and a visual feedback transmitted to the lower and upper sections via optical connection from FIG. 2A (Resistance Core) to Component 8B (Lower Section Light) and Component 8A (Upper Section Light). FIG. 4A (Smart Watch) then signals the user through either/or vibration, visual, and audio response when the training session is complete. User then detaches components 1C and 1D (Straps) from attachment mechanisms 11C and 11D. 1A and 1B (Straps) are then detached from release mechanisms 11A and 11B. User then detaches 1A (Lower Section) and 3A (Upper Section) from 2A (Resistance core). FIG. 2A (Resistance Core) is then attached to Component 13 (Recharging System) to recharge Component 12 (Power Storage) for future use. Prior to use, the user combines all sections of the wearable training device (If necessary based on model), then puts the assembled wearable training device on a limb using various attachment methods. The power supply of the wearable training device is activated through a wireless signal from the mobile device to the wearable training device. The user then selects a training program from the software application and sets the resistance to motion on the wearable training device accordingly. The user then moves through the training program indicated by the application. As the user moves through the technique indicated, the speed of motion, correct position, and repetitions are monitored through sensors, and then sent to the CPU which can activate haptic/visual/auditory responses through the remote device or wearable training device that indicate compliance or non-compliance. In addition, resistance to motion is also applied or absent based on whether the user is in the correct position or not. After the user completes the training routine, the wearable training device is then removed from the user and then all sections can be detached and the section containing the main power supply is placed on an induction or other type of charger to recharge power.

In the preferred embodiment, a sectioned wearable training device is provided which includes a central core section, an upper section that is attachable/detachable from the central core section, and a lower section that is attachable/detachable from the central core section. When sections are combined, for one use, the upper rigid section is attached to the leg (tibia and fibula) by first connecting means and the lower rigid section is connected to the thigh (femur) by a second connecting means. The core (or resistance hinge) section, when connected to the upper and lower sections, provides a controlled resistance to movement of the upper section in relation to the lower section. In addition, at least one of said sections includes, computer processing, sensing, and transmitting means for sending data to a device not physically attached to any other of the said sections, whereas said device is either a smart watch, smart phone, computer tablet, laptop, or other computing means. Said section also includes rechargeable power storage that allows for rapid recharging through means of an inductive or direct plug. Another aspect of the preferred embodiment includes the means to utilize a software application on the remote device which enables movement sensors on the core section to provide a signal which triggers a haptic and/or visual response through the remote device and/or the wearable training device. Said haptic/visual responses are determined by the user performing exercise techniques correctly or incorrectly as determined by certain sensed parameters including one or more of position, direction of movement, speed of movement, length of time, number of repetitions, tracking of correct and incorrect position, and other variables. In addition, said software application on the remote device or apparatus may configure and control the core sections resistance response to the position and orientation of user's limbs and body, along with the direction and speed of movement. The preferred embodiment also includes a means in which one section contains a slideable sleeve in so that the wearable training device can slide up and down the limb without rubbing against the skin while moving through the allowable range of motion. At least one of said sections can create a visual response when plugged into another section. Said sections are engaged through optical or electrical connectors.

In an alternate embodiment, as shown (on Sheet 3), 7A and 7B (Release Mechanisms) are thumb screws that screw into shell 4 at socket 4A and shell 6 and socket 6A respectively, effectively attaching FIG. 1A (Lower section) and FIG. 3A (Upper Section) to FIG. 2A (Resistance core). Another method of connecting FIGS. 1A and 3A to FIG. 2A by having a pull pin mechanism at position 7A and 7B, which is spring loaded to engage shell 6 at position 6A and shell 4 at position 4A by pulling up on spring loaded pull pins to slide shells into place and then releasing spring loaded pull-pins into sockets 4A and 6A, once again effectively attaches all sections. A third method of attaching FIGS. 1A and 3A to FIG. 2A involves 7A and 7B acting as magnets which are attracted to magnets embedded in the positions of 4A and 6A. When magnetic levers 7A and 7B are lifted up, these shells can move freely onto the resistance core, then the user releases magnets 7A and 7B which will then lock the shells into place through attraction of magnets located at 4A and 6A.

In the current embodiment (see Sheet 4) the resistance core (FIG. 2A) resists movements of crossbars 3A and 3B in relation to each other through resistance component 2A, which is comprised of friction-to-movement controlled by mechanical friction to movement. In a second method, the resistance core (FIG. 2A) resists movements of crossbars 3A and 3B in relation to each other through resistance component 2A, which is controlled by a spring or elastic band that increases in resistance as one crossbar moves in relation to the other, and then uses a clutch to disengage resistance caused by the spring or elastic band when the user reverses limb direction from flexion to extension. In a third method, the resistance core (FIG. 2A) resists movements of crossbars 3A and 3B in relation to each other through resistance component 2A, which uses an electrostatic resistance to movement that is controlled by the amount of electrical input into component 2A. In a fourth method, the resistance core (FIG. 2A) resists movements of crossbars 3A and 3B in relation to each other through resistance component 2A, which uses magnetics including rigid or fluidic elements that is controlled by the amount of electrical input into component 2A.

In the preferred embodiment, transmission between the resistance core and remote devices as noted (on Sheet 5), components 12 (electrical box) contains a wireless transmitter/receiver to send signals between the resistance core and remote devices such as a smart watch, smart phone, or other mobile devices. In the current embodiment, this is accomplished through the use of a Bluetooth transmitter/receiver.

A second embodiment includes transmission between the resistance core and remote devices would be through the use of another wireless communications protocol such as Wi-Fi. A third method of transmission would be through wireless RF (Radio Frequency) communication method. A fourth method of transmission between the resistance core and remote devices would be optical, such as infrared. And a fifth method of transmission between the resistance core and remote devices would be simple serial or USB point to point cable.

In the preferred embodiment, to prevent movement of apparatus against the skin while the user moves through the range of motion includes: As shown in Sheet 3, component 5 is a movable sleeve that rests securely against the user's limb while component 4 (shell) slides freely against sleeve 5 as the user moves through the range of motion.

In an alternative embodiment, a multicentric hinge is added to component 2A to allow crossbars 3A and 3B to adjust their position to counter movement of FIG. 3A (Upper section) as the user moves through the range of motion.

A third embodiment includes a hydraulic mechanism placed between crossbar 3A and shell 4 that allows an in-and-out movement of 3A in relation to 4 as the user moves through the range of motion.

To sensing leg position, the preferred embodiment is illustrated on Sheet 3, components 10A and 10B are used to determine position and orientation of the user and the user's limbs in relation to the rest of their body and ground. There are various sensors that can accomplish this. In the preferred embodiment we are using IMU sensors, (Inertial Measurement Units) which are comprised of accelerometers, magnetometers, and gyroscopes. A second embodiment of measuring movement would be through the use of potentiometers located within the component 2A that magnetically measures rotation of the hinge, in conjunction with inclinometers or other like sensors which provide information in relation to ground. A third method would be the use of optical sensors that tracks the relationship of crossbar 3B in relation to crossbar 3A, and the optical sensor would be within component 2A, in conjunction with inclinometers or other like sensors which provide information in relation to ground.

A fourth embodiment of tracking movement would be the use of remote camera sensing that senses body position, then sends a signal to the user's computing device, such as a smart watch, which in turn controls haptic/visual responses activated on either the resistance core or remote computing devices.

Different Resistance Cores and Recharging Methods. Resistance Cores can either have the sensing system or not depending whether movement tracking and user alerts are desirable or not. In the former, power would be required in the core. For powered cores, power can be provided to the exoskeleton core in various methods. In the preferred embodiment, the power storage, which is rechargeable, is located on FIG. 2A (Resistance Core), or it can also be located, through a wired connection, to a clip or box located on the user's hip using a belt. In this embodiment, the power storage unit is rechargeable through either a direct plug method into a standard electrical outlet or through inductive charging by placing the power storage unit on an inductive charging pad. In another method, power from the wearable training device is generated through movement of the hinge, which creates a generator motion to create its own power, which is then stored within a power storage device on the core. A third method of providing power to the wearable training device is by using linear induction, which generates electricity as the user walks, which is then stored in a power storage unit.

Electronic Use Management System. In the preferred embodiment, position and orientation sensors, communication subsystems, power subsystems, display elements, user interface elements and computational hardware and software are included that help to guide the user in the correct and most efficient use of the apparatus. In addition to the portions of the electronic use management system contained in the apparatus, external devices, such as smartwatches, smartphones, tablets and other computers, may be also be utilized to communicate to and interface with the user. This use management system can suggest and illustrate specific movements, in isolation or as a series, to guide the user in the use of the apparatus. Using its sensors, the system monitors and records the position and orientation of a user's upper and lower left leg in relationship to each other, the rest of the user's body and the ground. The user's movements are then compared to the suggested movements and feedback is provided to the user on their success in achieving key aspects of the suggested movements. In addition, the suggested number of repetitions of a given movement may be provided and feedback may be provided to the user on their achievement of the suggested number of repetitions. Feedback to the user can be provided by the use management system using visual, haptic or auditory means.

In the preferred embodiment, a smartwatch is, optionally, used as the main user interface and feedback element for the use management system. The smartwatch wirelessly communicates to the use management system using the Bluetooth LE communication protocol. The smartwatch is utilized to graphically communicate a series of suggested movements and the number of suggested repetitions for each movement to the user. The smartwatch provides feedback to the user on the successful completion of a given movement and tracks the number of successful and unsuccessful repetitions of a movement. The user is guided through successive movement types after successfully completing the suggested number of repetitions for a given movement. The user may choose a specific movement series or skip suggested movements using the smartwatches' touch-enabled graphical interface. Feedback on the successful completion of a given movement and the suggested number of repetitions of that movement is provided by the smartwatch using graphical, haptic and auditory means. This includes providing easily distinguishable haptic feedback on successful and unsuccessful movements using the smartwatches' haptic feedback system. Other embodiments can utilize other electronic devices, such as smartphones, tablets or other computers, communicating in a wired or wireless fashion, to perform the user interface and feedback functions of the smartwatch. Such functions can also be performed by the apparatus itself without the use of external devices. Additional embodiments augment or replace overall functionality by updating the software portions of the electronic use management system. Augmentations include: 1) the ability to save user performance data for review and analysis by the user or other persons including physical training & medical personnel; 2) the ability to provide real time performance data to persons, such as physical training and medical personnel, in addition to or instead of the user; and 3) the ability to stream or save performance data to the cloud or cloud-based fitness & health data sharing, monitoring & analysis services, systems & applications.

In the preferred method (see sheet 8) the rigid shell sections are offset from centerline of FIG. 2A (Resistance Core) to accommodate the offset of the tibia bone in relation to the femur bone when used on the leg. Another method involves offsetting the release mechanism (7B) on crossbar 3B to accommodate the offset angle of the tibia in relation to the femur. A third method of accommodating the natural offset of the tibia in relation to the femur would be to create a moldable aluminum connecting piece in which one end is connected to crossbar 3B and the other end is connected to shell 6, whereas the moldable aluminum connector can be molded in a custom manner to accommodate individual's exact offset.

Sheet 8 also shows the use of Magnetic Attachment Clips to attach the straps to the rigid shell sections. Once the length of the straps are initially set, the magnetic clips allow for quick attachment & detachment of the wearable training device from the user. It is also known in the art that other non-magnetic clips can be used, or both sides of the straps can just be slide through a slot and then through the use of Velcro, wrap back on themselves to secure the straps to the rigid shell sections.

From the above description, it can be understood that the wearable training device of this invention has several advantages, such as: (1) it can provide a programmed amount of precise resistance based on the user being in the correct position while doing an exercise, (2) can alert the user through haptic or visual responses as to if they are in the correct position or not while performing certain exercises, (3) the wearable training system can isolate key muscles while inhibiting other to influence the correct position of the pelvis, (4) the wearable training system can be easily attached using a magnetic attachment system, (5) the rigid shell sections of the wearable training system can be removed from the resistance hinge (or core section) to allow different rigid shell sections to be attached, allowing for multiple users without sharing components that touch the skin of different users, and (6) the wearable training system can wirelessly transmit data back and forth to remote devices during training to record various training data, and to provide the user with a software program and visual pictures to intelligently guide the user through a complete training program.

While the preferred embodiment of the invention has been described with some particularity, many modification and variations in the preferred embodiment can be made without deviating from the invention. Therefore, it can be understood that within the scope of the appended claims the invention can be practiced other than as specifically described.

What is claimed is:

1. An apparatus comprising; two or more sections that are easily attachable and detachable to each other. When sections are attached, said apparatus comprises an wearable device, in which at least one of said sections includes a resistance hinge means to allow at least 90 degrees range of motion in relation to at least one of other said sections. Said resistance hinge means providing resistance to movement of one section in relation to at least one of said other sections when attached. Said resistance hinge means being able to provide resistance to movement anywhere at least between 0.5 to 10 foot pounds in the flexion direction, and 0.2 to 1 foot pounds in the extension direction.

2. An apparatus as in claim 1 in which said resistance hinge means can be removed or attached.

3. An apparatus as in claim 1 in which one of said sections or resistance hinge means includes means for sensing position of the limb in relation to the rest of the body based on a software program that uses certain parameters including one or more of; limb position, direction of movement, speed of movement, length of time, number of repetitions, tracking of correct and incorrect position, and other variables to enable haptic responses to the user and record said variables.

4. An apparatus as in claim 1 in which at least one of said sections or resistance hinge means includes means for transmitting data to a device not attached to any of said other sections whereas said device receiving transmitted data is either a smart watch, smart phone, computer tablet, laptop, or other computing means.

5. An apparatus as in claim 1 in which at least one of said sections or resistance hinge means includes a means for power storage and computer processing.

6. An apparatus as in claim 3 in which said sensors provide input to a computer program that provides a haptic or visual response to the user based on various limb position parameters based on a preset computer program that controls a haptic or visual response to the user based on data received from one of said sections.

7. An apparatus as in claim 1 that uses magnetic retaining clips to secure straps, said clips attached to one or more ends of the straps, and to one or more of the sections, allowing for easy attachment and releasing of the strap to the section.

8. An apparatus as in claim 2 in which the resistance hinge means is controlled by a software program.

9. An apparatus as in claim 2 which includes a moveable drive means to vary the amount of resistance applied during motion.

10. A method of applying and using a neuromuscular training device comprising the steps of: applying to the leg a wearable device that includes an upper and lower section which is connected to a middle resistance core section, attaching the upper and lower rigid sections above and below the knee with straps or wearable material. Setting the amount of resistance to movement in the flexion direction to between 0.5 and 20 foot pounds, and setting resistance to movement in the extension direction between 0 and 5 foot pounds. Having the user flex and extend their leg while in certain positions.

11. A method as in claim 10 where prior to applying the device to the limb, the user assembles all sections of the device by easily attaching the upper and lower sections to a middle resistance core section, where the middle core section includes a method to provide resistance to movement in at least one direction of movement.

12. A method as in claim 10 comprising the steps placing the device only on the left leg of a user only, having the user move through certain neuromuscular training techniques while the device is on the left leg of the body only.

13. A method as in claim 10, whereas the training device includes limb position sensing means, computer processing means, and transmitting means. Having the user undergo certain movement techniques, sensing said movements, then transmitting said data to remote devices such as a smart watch, smart phone, computer tablets, or other remote devices not attached to the training device.

14. A method in accordance with claim 13, in which the remote device that receives data provides a haptic or visual response to the user based on a software program.

15. A method in accordance with claim 10 in which the amount of resistance to movement provided to the user is varied based on a software program based on the user's limb position.

16. A method in accordance with claim 10 where the user activates an application in a remote device that instructs the user through a progressive training program using haptic or visual cues.

17. A method in accordance with claim 10 in which power for the resistance core is provided through a rechargeable battery that is recharged by removing the device from the user and attaching the section containing the battery to an induction pad or other charging device.

18. A method of making a wearable training device from interchangeable sections comprising the steps of: attaching removable rigid structures to a connecting hinge means, attaching straps or wearable material to the removable rigid sections on each side of the hinges, and attaching a means to resist movement of the removable rigid structures in relation to each other.

19. A method in accordance with claim 18 that attaches computer processing, power storage, limb position sensing and signal transmission means to the wearable device.

20. An apparatus in accordance with claim 1 whereas the rigid sections are designed to only fit on the left leg of a user of the wearable device.
